(19) United States
(12) Patent Application Publication
(10) Pub. No.: US 2025/0093282 A1
(43) Pub. Date: Mar. 20, 2025

(54) SOIL SENSOR

(71) Applicant: DENSO CORPORATION, Kariya-city (JP)

(72) Inventors: Minekazu SAKAI, Kariya-city (JP); Naohisa NIIMI, Kariya-city (JP); Norihiro KURUMADO, Kariya-city (JP); Tomohide ARIKI, Kariya-city (JP); Kei SHIMAKURA, Kariya-city (JP); Keita NAMBARA, Kariya-city (JP); Masashi SUZUKI, Kariya-city (JP); Hidetada KAJINO, Kariya-city (JP); Naoki HASHIMOTO, Kariya-city (JP)

(21) Appl. No.: 18/968,547

(22) Filed: Dec. 4, 2024

Related U.S. Application Data
(63) Continuation of application No. PCT/JP2023/019595, filed on May 26, 2023.

(30) Foreign Application Priority Data
Jun. 6, 2022 (JP) 2022-091822

(51) Int. Cl. G01N 22/04; G01N 33/24

(52) U.S. Cl. CPC G01N 22/04; G01N 33/246

(57) ABSTRACT

A soil sensor includes a first signal line transmitting a high-frequency electromagnetic wave, and a porous body having a water permeability and a water retention property. The soil sensor further includes a second signal line transmitting a low-frequency electromagnetic wave and facing soil through the porous body, and a ground line arranged on an inside of the first signal line. The second signal line is arranged on an inside of the first signal line and the ground line. Thus, the first signal line and the second signal line are prevented from causing an influence to each other by an electric field generated by one of the two lines.

### CROSS REFERENCE TO RELATED APPLICATION

The present application is a continuation application of International Patent Application No. PCT/JP2023/019595 filed on May 26, 2023, which designated the U.S. and claims the benefit of priority from Japanese Patent Application No. 2022-091822 filed in Japan on Jun. 6, 2022. The entire disclosures of all of the above applications are incorporated herein by reference.

### TECHNICAL FIELD

The present disclosure herein relates to a soil sensor for detecting soil conditions.

### BACKGROUND

A relevant art discloses a soil sensor that simultaneously measures the water potential and volumetric water content of soil.

The soil sensor detects the volumetric water content and the water potential by switching between high-frequency and low-frequency modes using the same probe electrode. For example, when detecting in high-frequency mode, water content of a porous body is included in a detection area, thereby leading to a detection error in the water content of the soil to be detected.

### SUMMARY

A soil sensor of one exemplar of the present description includes a first signal line facing soil to transmit a high-frequency electromagnetic wave, a porous body having a water permeability and a water retention property, a second signal line facing the soil through the porous body to transmit a low-frequency electromagnetic wave having a frequency lower than the high-frequency wave and, and a ground line arranged on an inside of the first signal line. In addition, the second signal line is arranged on an inside of the first signal line and the ground line.

### BRIEF DESCRIPTION OF THE DRAWINGS

Objects, features, and advantages of the present disclosure will become more apparent from the following detailed description made with reference to the accompanying drawings, in which:

FIG. 1 is a control configuration diagram including a soil sensor according to a first embodiment;

FIG. 2 is a diagram showing a configuration of the soil sensor;

FIG. 3 is a schematic cross-sectional view showing a positional relationship between a first detector and a second detector of the soil sensor;

FIG. 4 is a diagram showing other example of a porous body of the first embodiment;

FIG. 5 is a diagram showing a configuration of a soil sensor according to a second embodiment;

FIG. 6 is a schematic cross-sectional view showing a positional relationship between a first detector and a second detector in the soil sensor;

FIG. 7 is a partial cross-sectional view of a base of a soil sensor according to a third embodiment;

FIG. 8 is a diagram showing a configuration of a soil sensor according to a fourth embodiment;

FIG. 9 is a diagram showing a first example of a soil sensor according to a fifth embodiment;

FIG. 10 is a diagram showing a second example of the soil sensor according to the fifth embodiment;

FIG. 11 is a diagram showing a third example of the soil sensor according to the fifth embodiment;

FIG. 12 is a diagram showing a fourth example of the soil sensor according to the fifth embodiment;

FIG. 13 is a diagram showing a fifth example of the soil sensor according to the fifth embodiment;

FIG. 14 is a diagram showing a sixth example of the soil sensor according to the fifth embodiment;

FIG. 15 is a partial cross-sectional view showing a sealing structure between a base and a case according to a sixth embodiment;

FIG. 16 is a cross-sectional view showing a support structure for a porous body according to a seventh embodiment;

FIG. 17 is a cross-sectional view showing a first example of a configuration of a base according to an eighth embodiment;

FIG. 18 is a cross-sectional view showing a second example of a configuration of the base according to the eighth embodiment;

FIG. 19 is a diagram illustrating an abnormality determination process for a soil sensor of a ninth embodiment;

FIG. 20 is a diagram illustrating an abnormality determination process for a soil sensor of a tenth embodiment; and

FIG. 21 is a diagram illustrating an abnormality determination process for a soil sensor of a tenth embodiment.

### DETAILED DESCRIPTION

It is an object of the present disclosure in the description to provide a soil sensor capable of reducing detection errors of soil water content.

Various aspects disclosed in the present description employ different technical means to achieve their respective objects.

A soil sensor according to one exemplar of the present description includes a first signal line facing soil to transmit a high-frequency electromagnetic wave, a porous body having a water permeability and a water retention property, a second signal line facing the soil through the porous body to transmit a low-frequency electromagnetic wave having a frequency lower than the high-frequency wave and, and a ground line arranged on an inside of the first signal line. In addition, the second signal line is arranged on an inside of the first signal line and the ground line.

In the soil sensor, the second signal line, through which a lower frequency electromagnetic wave is transmitted, is arranged on an inside of the first signal line, through which a high frequency electromagnetic wave is transmitted. Further, by arranging the ground line at a position between the first signal line and the second signal line, it is possible to suppress an influence of the electric field formed by the high-frequency electromagnetic wave on the second signal line. Moreover, an influence of the electric field formed by the low-frequency electromagnetic wave on the first signal line can be also restricted. Therefore, according to the above-described technical features, the soil sensor is capable of reducing detection errors in a soil water content.

Hereinafter, several embodiments for carrying out the present disclosure will be described with reference to the drawings. In each embodiment, parts corresponding to matters explained in the preceding embodiment may be given the same reference numerals, and duplicate explanations may be omitted. In case where only a part of the configuration is described in each embodiment, other embodiments previously-described may be applied to the other parts of the configuration. In addition to parts that are specifically described as combinable in each embodiment, it is also possible to combine other parts of the embodiments even if not expressly stated so, provided that there is no particular problem with such combination.

### First Embodiment

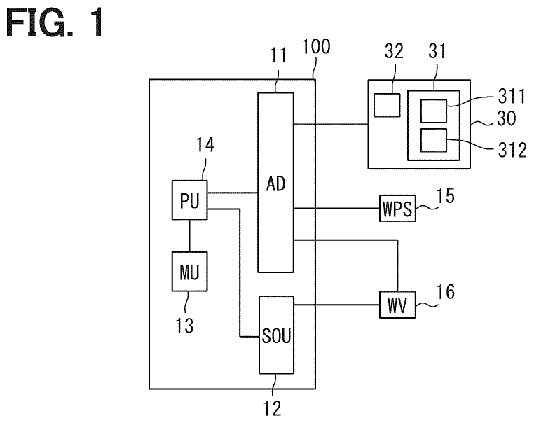

The first embodiment disclosing an example of a soil sensor will be described with reference to FIGS. 1 to 4. An irrigation system includes a watering device and a control device 100. The watering device supplies irrigation water to plants in a field. The watering device includes a pump, a water supply pipe, and the like. The pump acts as a water source that drives irrigation water down the supply pipes. When irrigation is performed, the control device 100 controls irrigation water based on a supply time and an amount of irrigation water supplied from the watering device to the plant. The control device 100 uses a detection value of a water pressure sensor (WPS) 15 to feedback control a valve opening degree of a water supply valve (WV) 16. The water supply valve 16 controls a flow rate of irrigation water discharged from the water supply pipe by having the valve opening controlled by the control device 100.

As shown in FIG. 1, the control device 100 includes an acquisition unit 11, a signal output unit 12, a memory unit 13, and a processing unit 14. In the drawing, the acquisition unit 11 is represented as AD, the signal output unit 12 is represented as SOU, the memory unit 13 is represented as MU, and the processing unit 14 is represented as PU. The acquisition unit 11 receives as input an environmental value detected by a soil sensor 30. The water pressure detected by the water pressure sensor 15 and the valve opening degree of the water supply valve 16 are input to the acquisition unit 11.

The signal output unit 12 is electrically connected to the water supply valve 16. A control signal for controlling the valve opening degree of the water supply valve 16 is output from the signal output unit 12 to the water supply valve 16.

The memory unit 13 is a non-transitory, substantial storage medium that non-temporarily stores programs and data that can be read by a computer or processor. The memory unit 13 includes a volatile memory and a non-volatile memory. The memory unit 13 stores a program for the processing unit 14 to execute arithmetic processing. The memory unit 13 temporarily stores data when the processing unit 14 executes arithmetic processing. The memory unit 13 stores various data input to the acquisition unit 11 and an acquisition time of the various data.

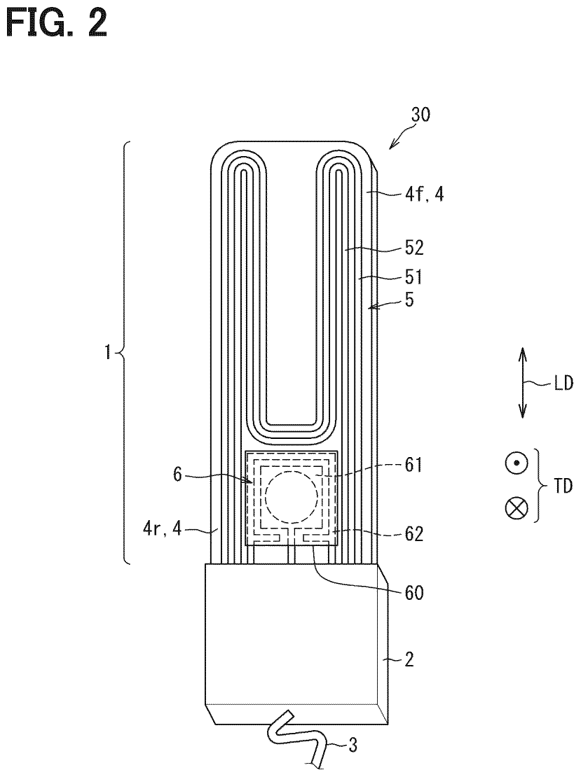

As shown in FIG. 2, the soil sensor 30 includes a detector 1 and a case 2. The detector 1 is a part that is buried in the soil of a field. The case 2 may be buried in the soil or installed outside the soil without being buried in the soil. The case 2 houses a circuit section including a CPU. The circuit section includes a processing unit 31 that performs arithmetic processing using data detected by the detector 1 of the soil sensor 30, and a communication unit 32 that communicates with the control device 100. The case 2 may be configured to include a thermistor capable of measuring temperature. The irrigation system that controls irrigation water for the soil monitors the soil water content using the soil sensor 30, and determines whether to supply irrigation water or stops irrigation water according to a detected value.

The detector 1 includes a first detector 5 and a second detector 6. The first detector 5 includes a first signal line 51 and a ground line 52. The second detector 6 includes a second signal line 61, a ground line 62, and a porous body 60. The detector 1 includes the first signal line 51, the second signal line 61, the porous body 60, and a base 4.

The circuit section built into the case 2 applies a frequency signal corresponding to a high frequency band within a predetermined range between an end of the first signal line 51 and an end of the ground line 52. The circuit section applies a frequency signal corresponding to a low frequency band within a predetermined range between an end of the second signal line 61 and an end of the ground line 62. The first signal line 51 transmits high-frequency electromagnetic waves such as microwaves or the like. The second signal line 61 transmits a low-frequency electromagnetic wave, which has a lower frequency than the electromagnetic wave transmitted through the first signal line 51.

The frequency of the electromagnetic waves transmitted through the first signal line 51 is preferably set to a frequency band that is 100 times or more higher than the frequency of the electromagnetic waves transmitted through the second signal line 61. A frequency relationship described above greatly contributes to preventing an influence between the electric field generated by one signal line and the electric field generated by the other signal line to each other.

The base 4 is formed, for example, from a water-impermeable material, and it is preferable that water does not penetrate into an inside of the base 4. At least a surface of the base 4 is an insulator to prevent short circuits between the first signal lines 51, between the second signal lines 61, and between the first signal line 51 and the second signal line 61.

The base 4 is made of, for example, acrylic or other resin material. The base 4 may be made of a metal such as aluminum, iron or the like, and a surface of the base may have an insulation treatment. The base 4 has a plate-like shape whose thickness is smaller than its width and length. The base 4 may be what is called a printed circuit board. Further, the shape of the base 4 may be other shapes.

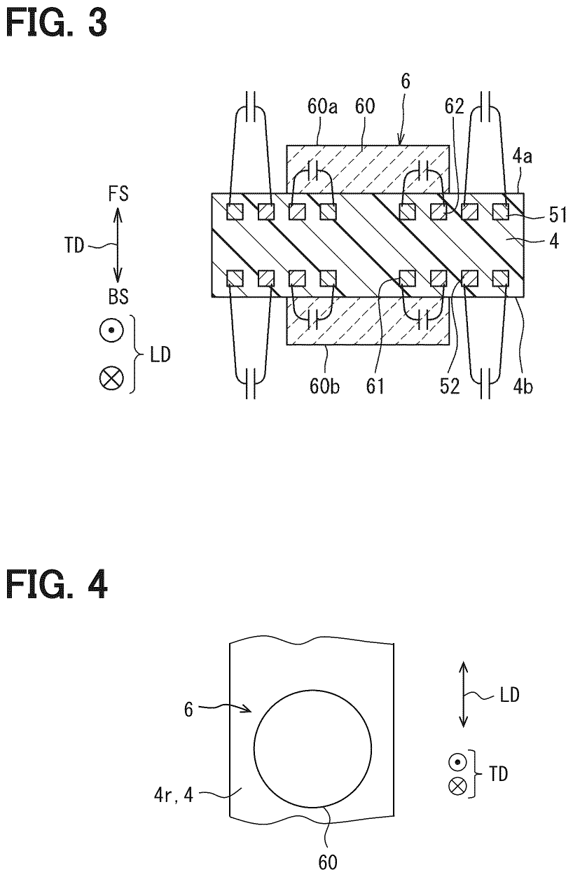

As shown in FIG. 3, the first signal line 51 and the ground line 52 are provided on one surface of the base 4 in a thickness direction TD. The first signal line 51 and the ground line 52 are provided on a surface of the base 4 on the other side in the thickness direction TD. The first signal line 51 and the ground line 52 are provided in the same configuration on both surfaces of the base 4 in the thickness direction TD. In FIG. 3, a front surface on one side in the thickness direction TD is denoted as FS, and a back surface on the other side is denoted as BS. The first signal line 51 is in direct contact with the soil. The first signal line 51 is provided on an outside of the ground line 52 so as to surround the ground line 52 on the outside thereof. The ground line 52 is provided to extend along the first signal line 51 at a constant interval on an inside of the first signal line 51.

The first signal line 51 and the ground line 52 include a folded line positioned at a tip 4f of the base 4 and a case-side line positioned at a root 4r of the base 4. The folded line and the case-side line are connected to form a continuous first signal line 51. The folded line forms a line that connects (a) a portion extending from the case to the tip and (b) a portion extending from the case to the tip. As shown in FIG. 2, the first signal line 51 includes a pair of folded lines adjacent to each other in a width direction of the base 4. The first signal line 51 includes a pair of case-side lines arranged in the width direction of the base 4. The width direction is a direction perpendicular to both the thickness direction TD and a longitudinal direction LD of the base 4. The first signal line 51 is formed in a loop shape on the front surface of the base 4, with a plurality of folded portions.

As shown in FIG. 3, the second signal line 61 and the ground line 62 are provided on a surface of the base 4 on the other side in the thickness direction TD. The second signal line 61 and the ground line 62 are provided on the surface of the base 4 on the other side in the thickness direction TD. The first signal line 51 and the ground line 52 are provided in the same configuration on both of the front and back surfaces of the base 4 in the thickness direction TD. The second signal line 61 and the ground line 62 are provided at a position between the pair of case-side lines.

The second signal line 61 is positioned on an inside of the first signal line 51. The second signal line 61 is surrounded by the first signal line 51 on an outside, and is provided on an inside of the first signal line 51. The ground line 52 and the ground line 62 are provided respectively at a position between the second signal line 61 and the first signal line 51. The second signal line 61 is surrounded by the ground line 62 on an outside, and is provided on an inside of the ground line 62. The second signal line 61 is surrounded by the ground line 52 on an outside and is provided on an inside of the ground line 52. The second signal line 61 is arranged at a position that can avoid an area filled with an electric field that is formed by the first signal line 51 and the ground line 52. Therefore, the second signal line 61 and the first signal line 51 are prevented from causing an influence to each other by an electric field generated by one of the two lines.

The second signal line 61 is covered with the porous body 60 in which a large number of pores are formed. The porous body 60 is made of a material having water permeability and water retention property. The porous body 60 is made of, for example, resin or ceramics. The second signal line 61 is in contact with the soil via the porous body 60. As shown in FIG. 3, the porous body 60 has a shape in which a surface 60a and a surface 60b facing each other in the thickness direction of the base 4 protrude from the base 4. The surface 60a on one side in the thickness direction is arranged at a position protruding from a surface 4a on one side of the base 4. The surface 60b on the other side in the thickness direction is arranged at a position protruding from a surface 4b on the other side of the base 4.

The first signal line 51 and the second signal line 61 are made of a material containing a metal such as gold, copper or the like. The first signal line 51 and the second signal line 61 are each formed in a ring shape provided along a periphery of the base 4. The first signal line 51 and the second signal line 61 are provided in pairs. The shapes and numbers of the first signal line 51 and the second signal line 61 are not limited to the above-described configuration. The first signal line 51 and the case 2 are electrically connected by a conductive wire. The second signal line 61 and the case 2 are connected by a conductive wire.

It is preferable that a surface shape of the porous body 60 exposed to the soil is a polygon having more sides than square. Further, as shown in FIG. 4, the surface of the porous body 60 exposed to the soil is preferably circular or cylindrical. When it is cylindrical, the porous body 60 has an outer circumferential surface that protrudes from an outer circumferential surface of the base 4. The porous body 60 having such a surface shape contributes to appropriate detection of a water potential WP, by improving adhesion to the soil and allowing the ceramics to smoothly absorb water from the soil.

It is preferable that the porous body 60 has a pore distribution suitable for the soil used for growing vegetables and the like. The porous body 60 having a pore distribution suited for such soil contributes to measuring the soil water potential WP with high accuracy. Therefore, the pore distribution formed in the porous body 60 is such that pores of various sizes within a range of 0.1 to 100 μm are evenly distributed in numbers.

The surface 4a and the surface 4b of the base 4 have projections and recesses formed thereon, each having a depth within a range of 0.1 to 500 μm. The projections and recesses with a depth close to 0.1 μm contributes to improvement of the adhesion between a clay soil and a printed circuit board. The projections and recesses with a depth of nearly 500 μm contributes to improvement of the adhesion between the sandy soil and the printed circuit board. The base 4 having the projections and recesses of such depth contributes to improvement of the adhesion between the soil and the printed circuit board, and enables appropriate detection of a sensing signal.

As shown in FIG. 1, the soil sensor 30 includes the processing unit 31 that includes a TDT measurement unit 311 and a capacitance measurement unit 312 and functions as a CPU, and the communication unit 32. The communication unit 32 outputs a processing result by the processing unit 31 to the acquisition unit 11 of the control device 100. The soil sensor 30 may be configured to include a CPU and a measuring device that function as the TDT measurement unit 311 and the capacitance measurement unit 312 by calculation using a program or the like. The processing unit 31 may perform detection using both of the signal line positioned on the surface 4a on one side and the signal line positioned on the surface 4b on the other side, or may perform detection using either one of them.

The TDT measurement unit 311 is connected to the first signal line 51 by a conductive wire. The TDT measurement unit 311 measures a dielectric constant of the soil using the first signal line 51 that transmits high-frequency electromagnetic waves. The TDT measurement unit 311 measures a transmission time of an electromagnetic wave transmitted through a signal line by a TDT method, also known as a time domain transmission method, to measure a relative dielectric constant of the soil. The TDT measurement unit 311 calculates a volumetric water content VWC using a correlation curve that specifies a relationship between the volumetric water content VWC of the soil and the dielectric constant or the relative dielectric constant. The correlation curve is a predetermined characteristic curve that is stored in advance in a memory unit or the like for each type of soils. The soil sensor 30 transmits the volumetric water content VWC to the control device 100 via the communication unit 32. For example, the control device 100 controls an irrigation time and an amount of irrigation water based on the volumetric water content VWC of the soil.

The processing unit 31 detects an electrical conductivity EC of the soil based on (a) a slope of a rising edge or (b) a magnitude of an amplitude of the waveform of the transmission signal transmitted through the first signal line 51 by the high-frequency electromagnetic wave. The soil sensor 30 transmits the detected electrical conductivity EC to the control device 100 via the communication unit 32. For example, the control device 100 estimates an amount of fertilizer in the soil based on the electrical conductivity EC and uses the estimated amount for the control of irrigation water.

The capacitance measurement unit 312 is connected to the second signal line 61 by a conductive wire. The capacitance measurement unit 312 measures a capacitance of the porous body 60 by using a low-frequency electromagnetic wave transmitted through the second signal line 61. The water potential WP of the soil and the water potential WP of the porous body 60 are equivalent due to water adsorption and capillary forces. The capacitance measurement unit 312 measures the water potential WP of the soil using the capacitance of the porous body 60, by utilizing such an equivalent relationship. When the soil water potential WP is low, a situation is that water is held in the soil with a strong force, making it difficult for plants to absorb water from the soil. The soil sensor 30 transmits the measured water potential WP to the control device 100 via the communication unit 32. For example, the control device 100 controls the irrigation time and the amount of irrigation water based on the water potential WP of the soil.

The control device 100 may control the amount of water or the amount of irrigated culture solution based on a pF value of the soil so that the soil has the pF value appropriate for the cultivated crop. The control device 100 may control the amount of water or the amount of irrigated culture solution based on the measured pF value of the soil, by periodically measuring the pF value of the soil using a timer.

The soil sensor 30 of the first embodiment includes the first signal line 51 that transmits high-frequency electromagnetic waves and faces the soil, and the porous body 60 that has water permeability and water retention property. The soil sensor 30 has (a) the second signal line 61 that (i) transmits low-frequency electromagnetic waves that are lower in frequency than high-frequency waves, and (ii) faces the soil through the porous body, and (b) the ground line that is arranged on an inside of the first signal line 51. The second signal line 61 is arranged on an inside of the first signal line 51 and the ground line.

In such a soil sensor 30, the second signal line 61 for transmitting a lower frequency electromagnetic wave is arranged on an inside of the first signal line 51 for transmitting a high frequency electromagnetic wave. Further, by arranging the ground line at a position between the first signal line 51 and the second signal line 61, the influence of the electric field caused by the high-frequency electromagnetic waves on the second signal line 61 is suppressible. Moreover, the influence of the electric field caused by the low-frequency electromagnetic waves on the first signal line 51 is also suppressible. With such technology, the soil sensor 30 achieves a reduction of detection error in detecting the soil water content.

The processing unit 31 measures the volumetric water content of the soil using a transmission signal transmitted through a first signal line transmitted as a high-frequency electromagnetic wave, and measures the water potential of the soil using a low-frequency electromagnetic wave transmitted through a second signal line. In such manner, it is possible to suppress the mutual influence of the electric field generated by one signal line and the electric field generated by the other signal line, thereby providing a sensor that can measure both of the volumetric water content and the water potential.

Further, the processing unit 31 measures the electrical conductivity of the soil based on the waveform of the transmission signal transmitted through the first signal line transmitted as the high-frequency electromagnetic wave. In such manner, it is possible to provide a sensor that is capable of appropriately measuring the volumetric water content, the water potential, and the electrical conductivity.

### Second Embodiment

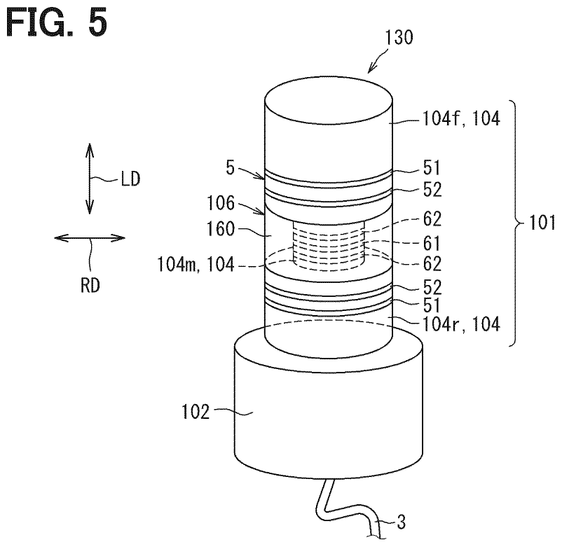

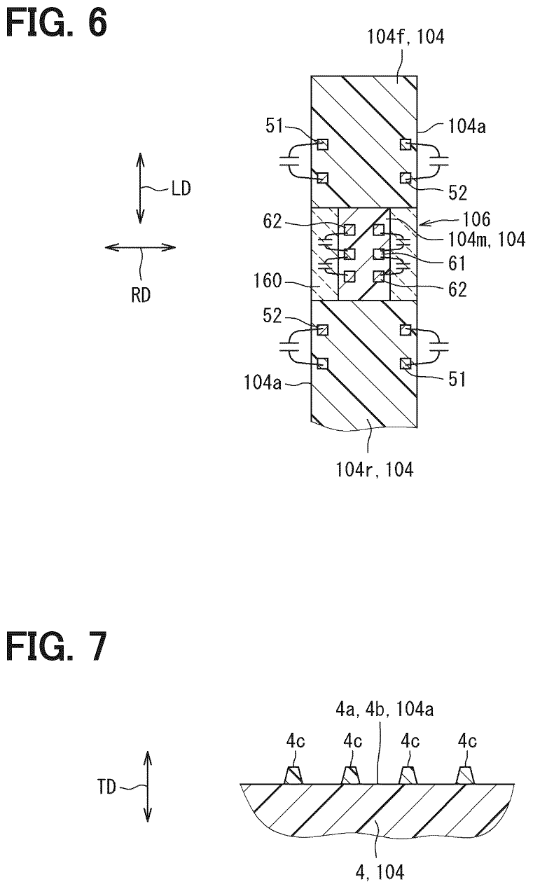

The second embodiment will be described with reference to FIGS. 5 to 6. A soil sensor 130 of the second embodiment differs from the first embodiment in the configurations of the base and the porous body. The configurations, operations, and effects of the second embodiment that are not specifically described are the same as those of the previously-described embodiment, and the differences will be mainly described in the following.

The soil sensor 130 shown in FIG. 5 includes a detector 101 and a case 102. The detector 101 has a cylindrical shape, and is buried in the soil of the field. The case 102 may be buried in the soil, or may be installed outside the soil without being buried in the soil. The detector 101 includes a first detector 105 and a second detector 106. The second detector 106 includes a second signal line 61, a ground line 62, and a porous body 160. The detector 101 includes a first signal line 51, a second signal line 61, a porous body 160, and a base 104.

The porous body 160 corresponds to a porous body 60 in the first embodiment. The base 104 is formed, for example, from a water-impermeable material, and it is preferable that water does not penetrate into an inside of the base 104. At least a surface of the base 104 is an insulator to prevent short circuits between the first signal lines 51, between the second signal lines 61, and between the first signal line 51 and the second signal line 61. The base 104 is formed from the same material and has the same surface treatment as the base 4 of the first embodiment. The base 104 has an axial direction of a column shape body as its longitudinal direction LD.

As shown in FIG. 6, the first signal line 51 and the ground line 52 are provided on a peripheral surface of a tip 104f of the base 104, each of which has an annular shape. The first signal line 51 and the ground line 52 are provided on a peripheral surface of a root 104r of the base 104, each of which has an annular shape. With such a configuration, the soil sensor 130 can detect the volumetric water content VWC and the electrical conductivity at two positions spaced apart in the axial direction of the detector 101.

The tip 104f and the root 104r are formed to have the same dimension as an outer diameter. The first signal line 51 formed on the tip 104f and the first signal line 51 formed on the root 104r have the same radial position on the base 104. The first signal line 51 on a tip 104f side and the first signal line 51 on a root 104r side are in direct contact with the soil. The ground line 52 is provided on the tip 104f or on the root 104r at a position closer to the porous body 160 than the first signal line 51. The ground line 52 is provided along the first signal line 51 at a fixed interval therefrom at a position closer to the porous body 160 than the first signal line 51.

As shown in FIGS. 5 and 6, the second signal line 61 and the ground line 62 are provided on a surface of an intermediate portion 104m of the base 104. The intermediate portion 104m has a columnar body formed to have an outer diameter smaller than the tip 104f and the base 104r. The porous body 160 has an outer circumferential surface that is flush with an outer circumferential surface 104a of the base 104, and covers the second signal line 61 and the ground line 62. The porous body 160 may be configured to have an outer circumferential surface that protrudes from the outer circumferential surface 104a of the base 104. The ground line 62 is provided respectively at a position closer to the tip 104f and a position closer to the root 104r than the second signal line 61.

The second signal line 61 is positioned on a radial inside of the first signal line 51. The second signal line 61 constitutes a circular line that is spaced apart in the axial direction from the first signal line 51 and has a smaller diameter than the first signal line 51. The second signal line 61 is surrounded from a radial outside RD by the first signal line 51 provided on the tip 104f that is spaced apart therefrom in the axial direction. The second signal line 61 is surrounded from a radial outside RD by the ground line 52 provided on the tip 104f that is spaced apart therefrom in the axial direction. The second signal line 61 is surrounded from a radial outside RD by the first signal line 51 provided on the root 104r that is spaced apart therefrom in the axial direction. The second signal line 61 is surrounded from a radial outside RD by the ground line 52 provided on the root 104r that is spaced apart therefrom in the axial direction.

The second signal line 61 and the ground line 62 are covered with the porous body 160 in which a large number of pores are formed. The porous body 160 is a cylindrical body made of a material having water permeability and water retention property. The porous body 160 is made of, for example, ceramics. The second signal line 61 faces the soil via the porous body 160.

The first signal line 51 and the second signal line 61 are formed from a material containing a metal such as gold, copper or the like. The first signal line 51 and the second signal line 61 are each formed in a ring shape provided on a peripheral surface of the base 104. The first signal line 51 and the second signal line 61 are respectively provided in pairs. The shapes and numbers of the first signal lines 51 and the second signal lines 61 are not limited to the above-described configuration. The first signal line 51 and the case 2 are electrically connected by a conductive wire. The second signal line 61 and the case 2 are connected by a conductive wire.

It is preferable that the porous body 160 has a pore distribution suitable for the soil used for growing vegetables and the like. The porous body 160 having a pore distribution suited for such soil contributes to measuring the soil water potential WP with high accuracy. Therefore, the pore distribution formed in the porous body 160 is such that pores of various sizes within a range of 0.1 to 100 μm are evenly distributed in numbers.

An outer peripheral surface 104a of the base 104 have projections and recesses formed thereon, each having a depth within a range of 0.1 to 500 μm. The projections and recesses with a depth close to 0.1 μm contributes to improvement of the adhesion between the clay soil and the printed circuit board. The projections and recesses with a depth close to 500 μm contributes to improvement of the adhesion between the sandy soil and the printed circuit board. The base 104 having the projections and recesses of such depth contributes to improvement of the adhesion between the soil and the printed circuit board, and enables appropriate detection of a sensing signal.

### Third Embodiment

The third embodiment will be described with reference to FIG. 7. A soil sensor of the third embodiment differs from the above-described embodiments in the configuration of a surface of a base. The configurations, operations, and effects of the third embodiment not specifically described are similar to those of the previously-described embodiments, and only the differences will be described in the following.

As shown in FIG. 7, the soil sensor of the third embodiment has a plurality of protrusions 4c that protrude from the surface of a base 4, 104. The plurality of protrusions 4c protrude from at least one of a surface 4a on one side and a surface 4b on the other side of the base 4, 104. The plurality of protrusions 4c are formed by dropping molten resin onto the surface 4a on one side and the surface 4b on the other side and then solidifying the molten resin.

According to the soil sensor of the third embodiment, the plurality of protrusions 4c contribute to improvement of holdability of the soil sensor in the soil and to improvement of the ease of installation work of the soil sensor.

### Fourth Embodiment

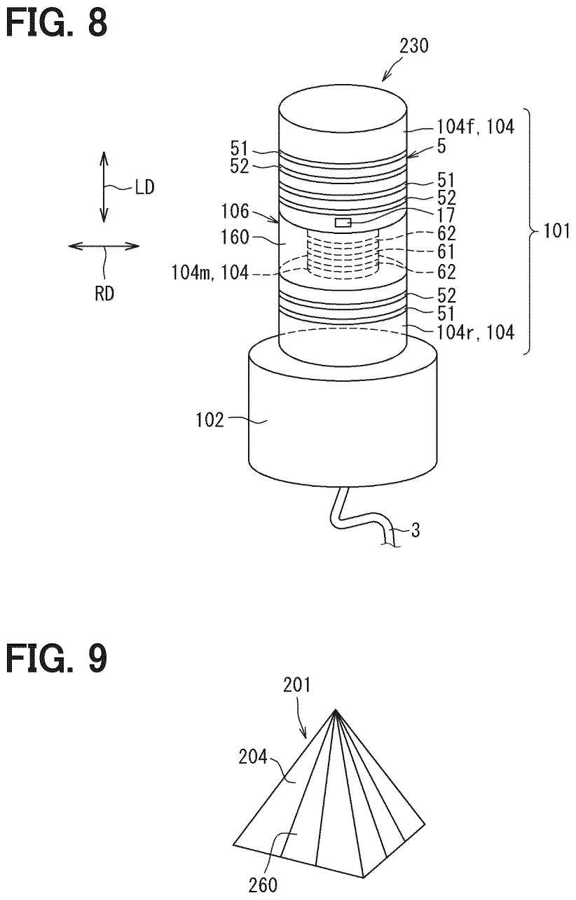

The fourth embodiment will be described with reference to FIG. 8. A soil sensor 230 of the fourth embodiment differs from the second embodiment in the configuration of a first detector. The configurations, operations, and effects of the fourth embodiment that are not specifically described are similar to those of the previously-described embodiments, and only the differences will be described in the following.

As shown in FIG. 8, the soil sensor 230 includes a greater number of ring-shaped first signal lines 51 than in the second embodiment. A detector 101 of the soil sensor 230 includes a first signal line 51, a second signal line 61, a porous body 160 and a base 104. The base 104 corresponds to the base 4 in the first embodiment. A second detector 106 includes the second signal line 61, a ground line 62, and the porous body 160.

The base 104 is provided with a plurality of ring-shaped first signal lines 51 and a plurality of ring-shaped ground lines 52. The first signal lines 51 are arranged at a plurality of spaced apart positions on the base 104. A plurality of first signal lines 51 are provided on a tip 4f of the base 4, which are spaced apart from each other in an axial direction or in a longitudinal direction LD. A plurality of ground lines 52 are provided on the tip 4f, which are spaced apart from each other in the axial direction or in the longitudinal direction LD. The ground line 52 is provided at a position between the two first signal lines 51 spaced apart from each other on the tip 4f. A temperature sensor 17 for detecting temperature of the soil is provided on the tip 4f. Such a configuration makes it possible to provide the soil sensor 230 that includes all of a temperature sensor, a water potential detector, and TDT-type detectors at multiple positions. The soil sensor 230 has achieved a volume reduction of the device and a simultaneous detection of multiple methods, by having an integrated configuration of multiple-type detection functions.

### Fifth Embodiment

The fifth embodiment will be described with reference to FIGS. 9 to 14. A soil sensor of the fifth embodiment differs from the above-described embodiments in the shape and configuration of a base and a porous body. The configurations, operations, and effects of the fifth embodiment not specifically described are the same as those of the previously-described embodiments, and only the differences will be described in the following.

As shown in FIGS. 9 to 13, in the soil sensor of the fifth embodiment, the structure forming the porous body and the base, or the structure forming the first detector and the second detector, is a regular polyhedron. The porous body and the base shown in FIGS. 9 to 14 are each made of the same material as in the first embodiment.

FIG. 9 shows a first example of the soil sensor according to the fifth embodiment. In the soil sensor shown in FIG. 9, a detector 201 is formed in a regular tetrahedral shape. The detector 201 includes a base 204 and a porous body 260. The base 204 has a first signal line 51 and a ground line 62 respectively arranged over a wide surface area of the base 204 for those lines to extend along the surface thereof. The porous body 260 covers a second signal line 61, with its surface flush with a surface of the base 204 or protruding from the surface of the base 204. The second signal line 61 is arranged on an inside of the first signal line 51 and the ground line 62 in the detector 201. According to the soil sensor shown in FIG. 9, it has a symmetrical shape, thereby evenly bearing external stress, ensuring strength, and not easily breakable.

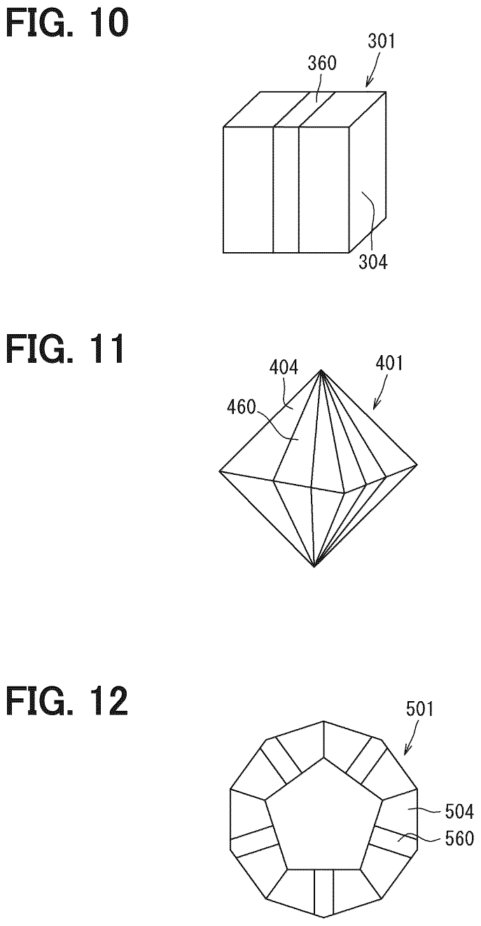

FIG. 10 shows a second example of the soil sensor according to the fifth embodiment. The soil sensor shown in FIG. 10 has a detector 301 formed in a regular hexahedron shape. The detector 301 includes a base 304 and a porous body 360. The base 304 has a first signal line 51 and a ground line 62 respectively arranged over a wide surface area of the base 304 for those lines to extend along the surface thereof. The porous body 360 covers a second signal line 61, with its surface flush with a surface of the base 304 or protruding from the surface of the base 304. The second signal line 61 is arranged on an inside of the first signal line 51 and the ground line 62 in the detector 301. According to the soil sensor shown in FIG. 10, it has a symmetrical shape, thereby evenly bearing external stress, ensuring strength, and not easily breakable.

FIG. 11 shows a third example of the soil sensor according to the fifth embodiment. The soil sensor shown in FIG. 11 has a detector 401 formed in a regular octahedron shape. The detector 401 includes a base 404 and a porous body 460. The base 404 has a first signal line 51 and a ground line 62 respectively arranged over a wide surface area of the base 404 for those lines to extend along the surface thereof. The porous body 460 covers a second signal line 61, with its surface flush with a surface of the base 404 or protruding from the surface of the base 404. The second signal line 61 is arranged on an inside of the first signal line 51 and the ground line 62 in the detector 401. According to the soil sensor shown in FIG. 11, it has a symmetrical shape, thereby evenly bearing external stress, ensuring strength, and not easily breakable.

FIG. 12 shows a fourth example of the soil sensor according to the fifth embodiment. The soil sensor shown in FIG. 12 has a detector 501 formed in a regular dodecahedron shape. The detector 501 includes a base 504 and a porous body 560. The base 504 has a first signal line 51 and a ground line 62 respectively arranged over a wide surface area of the base 504 for those lines to extend along the surface thereof. The porous body 560 covers a second signal line 61, with its surface flush with a surface of the base 504 or protruding from the surface of the base 504. The second signal line 61 is arranged on an inside of the first signal line 51 and the ground line 62 in the detector 501. According to the soil sensor shown in FIG. 12, it has a symmetrical shape, thereby evenly bearing external stress, ensuring strength, and not easily breakable.

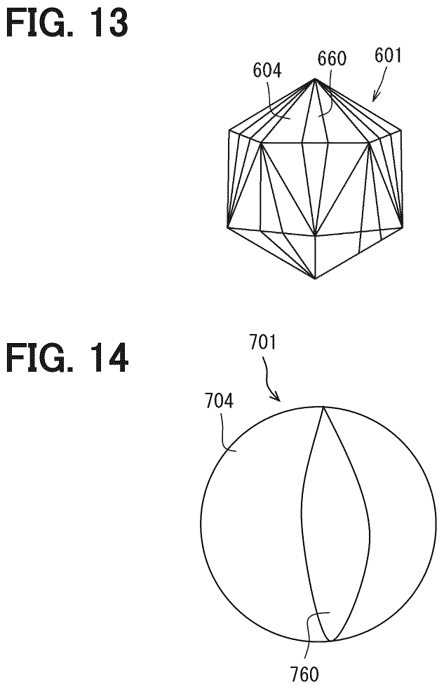

FIG. 13 shows a fifth example of the soil sensor according to the fifth embodiment. The soil sensor shown in FIG. 13 has a detector 601 formed in a regular icosahedral shape. The detector 601 includes a base 604 and a porous body 660. The base 604 has a first signal line 51 and a ground line 62 respectively arranged over a wide surface area of the base 604 for those lines to extend along the surface thereof. The porous body 660 covers a second signal line 61, with its surface flush with a surface of the base 604 or protruding from the surface of the base 604. The second signal line 61 is arranged on an inside of the first signal line 51 and the ground line 62 in the detector 601. According to the soil sensor shown in FIG. 13, it has a symmetrical shape, thereby evenly bearing external stress, ensuring strength, and not easily breakable. FIG. 14 shows a sixth example of the soil sensor according to the fifth embodiment. As shown in FIG. 14, the soil sensor may have a configuration in which a detector 701 is formed in a spherical shape. The base 704 has a first signal line 51 and a ground line 62 respectively arranged over a wide surface area of the base 704 for those lines to extend along the surface thereof. The porous body 760 covers a second signal line 61, with its surface flush with a surface of the base 704 or protruding from the surface of the base 704. The second signal line 61 is arranged on an inside of the first signal line 51 and the ground line 62 in the detector 701. According to the soil sensor shown in FIG. 14, it has a symmetrical shape, thereby evenly bearing external stress, ensuring strength, and not easily breakable.

### Sixth Embodiment

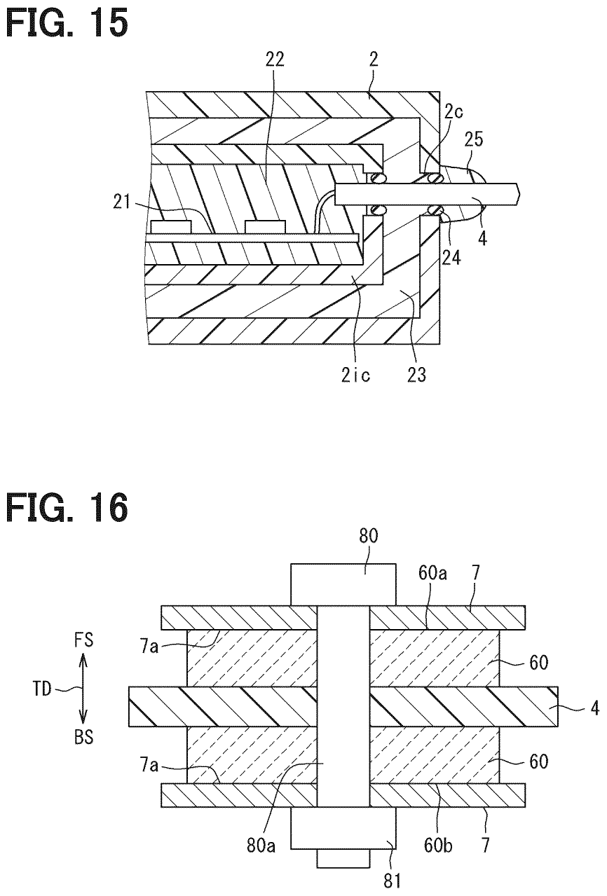

The sixth embodiment will be described with reference to FIG. 15. A soil sensor of the sixth embodiment differs from the above-described embodiments in the support structure of a base 4 relative to a case 2. The configurations, operations, and effects of the sixth embodiment that are not specifically described are the same as those of the previously-described embodiments, and only the differences will be described in the following.

As shown in FIG. 15, the soil sensor of the sixth embodiment includes a seal supporter 25 that supports the base 4 when inserted into an inside of the case 2 and functions as a seal structure. The base 4 has a portion that is inserted into an opening 2c of the case 2 and is arranged on an inside of the case 2, and a portion that protrudes to an outside of the case 2. A gap between the base 4 and the opening 2c of the case 2 is sealed by a sealer 24 such as a packing or the like.

The case 2 further has an inner case 2ic in an inside thereof. In other words, the case 2 has a double structure with the inner case 2ic built therein, and the case 2 serves as an outer case. The inner case 2ic accommodates a substrate 21 including a circuit section. The substrate 21 and a pattern substrate of the base 4 are electrically connected by a cable. When the base 4 is inserted into an opening 2c on the inner case 2ic, the gap between the base 4 and the opening 2c of the case 2 is sealed by a sealer such as a packing or the like. This sealer and the sealer 24 have the function of supporting the base 4. The inside of the inner case 2ic is filled with a potting 22 such as urethane resin or the like. A space between the case 2 and the inner case 2ic is filled with a water-absorbent material 23 made of a resin material having water-absorbency. The water absorbent material 23 may be made of, for example, polyacrylate. Even if water penetrates into the inside of the case 2, the water absorbent material 23 prevents water from entering the circuit section, thereby contributing to improving the waterproofing of the circuit.

The seal supporter 25, formed in a fillet shape on an outside of the case 2, adheres to (a) a surface of a part of the base 4 that protrudes to an outside from the case 2 and (b) a surface of the case 2. The seal supporter 25 is formed with an elastic material that is deformable when subjected to an external force. The seal supporter 25 is preferably made of a urethane resin having high viscosity. The high viscosity of the seal supporter 25 enables it to enhance adhesion (a) to the surface of the base 4 and (b) to the opening 2c of the case 2. In such manner, the seal supporter 25 provides the function of supporting the base 4 relative to the case 2 and the function of preventing water from entering the case 2.

### Seventh Embodiment

The seventh embodiment will be described with reference to FIG. 16. A soil sensor of the seventh embodiment differs from the above-described embodiments in the support structure for a porous body and a base. The configurations, operations, and effects of the seventh embodiment that are not specifically described are the same as those of the previously-described embodiments, and only the differences will be described in the following.

As shown in FIG. 16, the soil sensor of the seventh embodiment has a configuration in which a porous body 60 and a base 4 are sandwiched by an outer supporter 7. The outer supporter 7 is a plate-like part that faces the soil, and is made of, for example, stainless steel. A shaft portion 80a of a bolt 80 penetrates a laminated structure which is made up of the outer supporter 7, the porous body 60, the base 4, the porous body 60 and the outer supporter 7 laminated in the written order. In this sandwich configuration, the base 4, the porous bodies 60 arranged on both sides of the base 4, and the outer supporters 7 arranged on an outside of each porous body 60 are restrained by the bolt 80 and a nut 81. The bolt 80 and the nut 81 are an example of a restraining member that restrains the laminated structure in order to compress it.

A surface of the outer supporter 7 has a flatness such that no gap exceeding 2 mm occurs between the outer supporter 7 and the porous body 60. Such configuration makes it possible to prevent soil particles from entering between the outer supporter 7 and the porous body 60 when the laminated structure is placed in the soil. In such manner, the porous body 60 is prevented from cracking.

### Eighth Embodiment

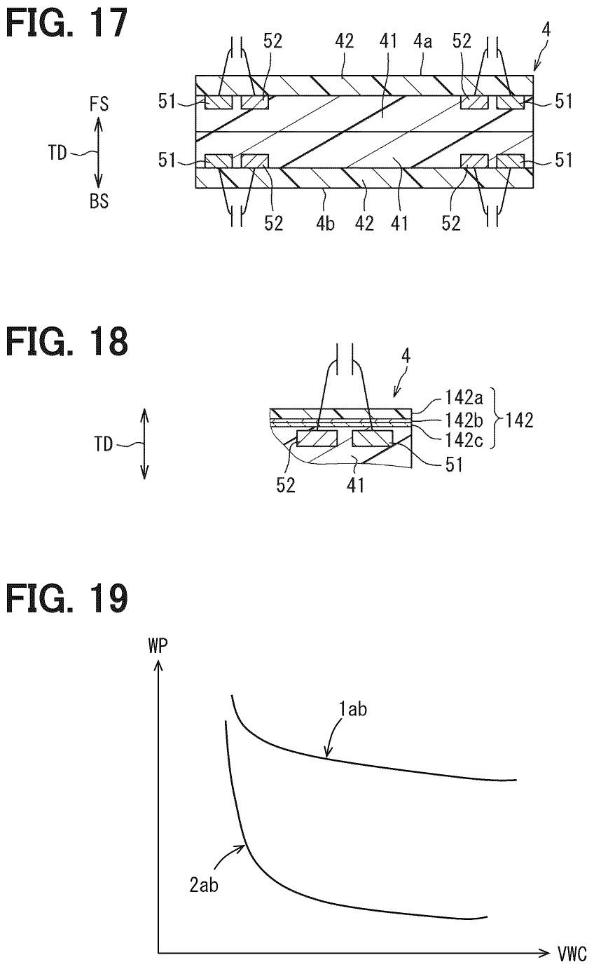

The eighth embodiment will be described with reference to FIGS. 17 and 18. A soil sensor of the eighth embodiment differs from the above-described embodiments in the configuration of a surface of a base. The configurations, operations, and effects of the eighth embodiment that are not specifically described are the same as those of the previously-described embodiments, and only the differences will be described in the following.

As shown in a first example of FIG. 17, the soil sensor of the eighth embodiment includes a base 4 having a plurality of layers. The base 4 of the eighth embodiment includes a plurality of signal layers 41 on which first signal lines 51 and ground lines 52 are arranged, and a surface layer 42 laminated on an outer side of each signal layer 41. The surface layer 42 is provided to form each of both surfaces of the base 4 in the thickness direction TD. The surface layer 42 is formed from a resin. The signal layer 41 is at least partially insulated to prevent a short circuit between the first signal lines 51. For example, when the thickness of the base 4 is 1.6 mm, the thickness of the surface layer 42 is set to a value included in a range of 200 to 400 μm. According to the first example, it is possible to prevent a pattern formed on the base 4 from coming into contact with the soil and being damaged, and it is possible to provide a soil sensor that is highly robust against the soil.

As shown in a second example of FIG. 18, the soil sensor of the eighth embodiment includes a surface layer 142 made up of a plurality of layers laminated on the surface of the base 4. The surface layer 142 includes an outermost resin layer 142a, a glass layer 142b positioned inside the resin layer 142a, and a ceramic layer 142c positioned inside the glass layer 142b. The resin layer 142a is made of a polymer resin, and has a thickness of, for example, about 10 μm. The glass layer 142b is a layer having a higher hardness than the resin layer 142a, and is formed to have a thickness of 0.1 to 0.3 μm. The ceramic layer 142c is a porous layer.

The surface layer 142 includes a plurality of layers laminated from the outside to the inside, and has the glass layer 142b or the ceramic layer 142c on the inside of the outermost resin layer 142a. With such configuration, the glass layer 142b and the like prevent a pattern formed on the inside from coming into contact with the soil and being damaged, thereby providing a soil sensor that is highly robust against the soil.

### Ninth Embodiment

The ninth embodiment will be described with reference to FIG. 19. A soil sensor of the ninth embodiment differs from the above-described embodiments in that an abnormality determination process is performed on the soil sensor. The configurations, operations, and effects of the ninth embodiment that are not specifically described are similar to those of the previously-described embodiments, and only the differences will be described in the following.

The soil sensor of the ninth embodiment performs an abnormality determination process using a characteristic curve of a volumetric water content VWC and a water potential WP of the soil shown in FIG. 19. FIG. 19 is an anomaly determination curve in which the horizontal axis represents the volumetric water content VWC of the soil, and the vertical axis represents the water potential WP. A curve 1ab in FIG. 19 is a boundary line indicating that the soil contains a lot of water but the porous body cannot absorb the water, which is an abnormal state that an adhesion of the sensor to the soil is poor. A curve 2ab in FIG. 19 is a boundary line indicating that water accumulates in a gap between the porous body and the soil to soak the sensor, which is an abnormal state in which the soil is dry. The abnormality determination curve is a predetermined characteristic curve that is stored in advance in a memory unit or the like of the soil sensor.

A processing unit 31 determines a normal state when the measured volumetric water content VWC and the water potential WP indicate a point between the curve 1ab and the curve 2ab. The processing unit 31 determines an abnormal state when the measured VWC and WP indicate a point not positioned in between the curve 1ab and the curve 2ab.

VWC sensors use a radio frequency electric field radiated into the soil to measure the VWC over the entire area covered by the electric field. Therefore, even if the VWC sensor does not adhere to the soil, the measurement value does not change significantly. On the other hand, the measurement value of the WP sensor is significantly affected by the quality of its adhesion to the soil. By combining the measurement values from these two types of sensors to plot a soil water retention curve, poor adhesion will be identified as a point that deviates from the normal state, making it possible to distinguish the poor adhesion from dry soil. Therefore, by using the curves 1ab and 2ab, it becomes possible to appropriately perform an abnormality determination, which establishes distinction between poor adhesion of the sensor to the soil and dry soil.

The processing unit 31 determines whether or not an abnormal state exists by using (a) the measured volumetric water content and water potential, and (b) pre-stored two abnormality determination curves that indicate the relationship between the volumetric water content and the water potential. In such manner, it is possible to distinguish poor adhesion of the sensor to the soil from dry soil. Thus, problems such as misunderstanding poor adhesion as dry soil and excessive watering are preventable, which then facilitate correction of sensor installation state.

### Tenth Embodiment

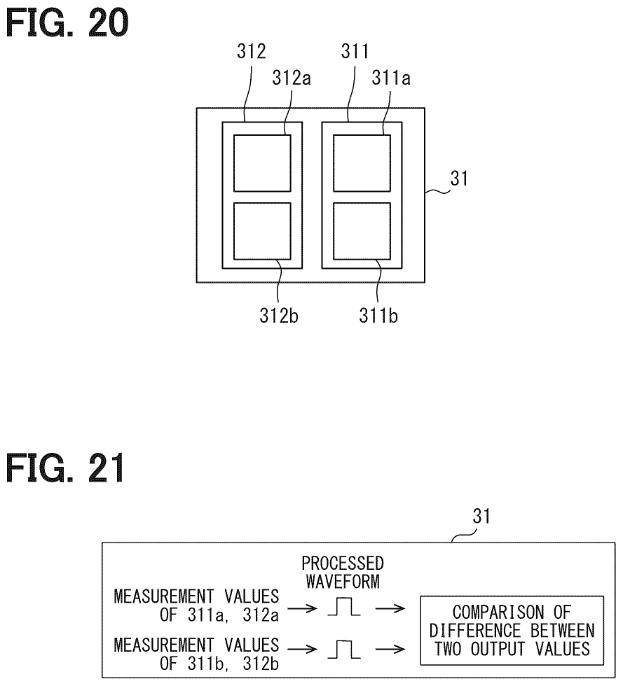

The tenth embodiment will be described with reference to FIG. 20. A soil sensor of the tenth embodiment differs from the above-described embodiments in that it performs an abnormality determination process based on a comparison of measurement values at two positions. The configurations, operations, and effects of the tenth embodiment that are not specifically described are the same as those of the previously-described embodiments, and only the differences will be described in the following.

As shown in FIG. 20, a TDT measurement unit 311 of the tenth embodiment includes a first measurement unit 311a and a second measurement unit 311b. The first measurement unit 311a performs measurement using a signal line arranged at a first position in a detector. The second measurement unit 311b performs measurement using a signal line arranged at a second position in the detector.

A capacitance measurement unit 312 of the tenth embodiment includes a first measuring unit 312a and a second measuring unit 312b. The first measuring unit 312a performs measurement using a signal line arranged at a first position in the detector. The second measuring unit 311b performs measurement using a signal line arranged at a second position different from the first position. A processing unit 31 determines whether or not a measurement value is abnormal based on a result of comparing the value measured at the first position with the value measured at the second position. For example, as shown in FIG. 21, the processing unit 31 determines whether or not there is an abnormality, by generating each processed waveform using two measurement values and analyzing a difference between these two output values. An example of the first position is a signal line arranged on one surface of a detector 1 on one side thereof in the thickness direction TD. An example of the second position is a signal line arranged on a back surface of the detector 1 on the other side in the thickness direction TD. In the above-described example, the processing unit 31 determines whether or not the measurement value is abnormal based on a result of comparing values measured independently on both sides of the detector 1.

The processing unit 31 adopts, as a comparison result of the measurement values at two positions described above, a difference of the measurement values, a difference in measured waveform such as shapes, widths, cycle times and the like, and determines a degree of deviation of such difference from a reference. Such a determination is made taking into consideration detection accuracy and processing accuracy. In comparing the measurement values at two positions, the processing unit 31 determines that an abnormality exists in the following example cases.

If one displacement between the measurement values at one position is too different apart from the other displacement at the other position, an abnormality is determined. If one of the measurement values remains constant and does not change, it is determined that an abnormality exists. If one measurement value is a positive value and the other continuous value is a negative value, an abnormality is determined. If a difference between the measured waveforms at the two positions is beyond an expected phase range or fluctuation range, an abnormality is determined. If the measurement values at two points during a self-diagnosis time deviate from the expected values, an abnormality is determined.

The processing unit 31 of the tenth embodiment determines whether or not an abnormal state exists based on the comparison result of the measurement values at two different positions. In such manner, the probability of detecting damage or broken line in the soil improves, enabling stable operation with appropriate output values and contributing to an improvement of the stability of irrigation systems that use soil sensors. Further, analyzing failures in soil sensors is made easy, by making it possible to detect failures due to factors such as self-heating, temperature changes and the like.

### Other Embodiments

The present disclosure in the description is not limited to the above-illustrated embodiments. The present disclosure encompasses the illustrated embodiments and variations by those skilled in the art based on those embodiments. For example, the present disclosure is not limited to the combinations of parts and elements shown in the embodiments, and can be implemented in various modified forms. The present disclosure can be implemented in various combinations. The present disclosure may have additional portions that can be added to the embodiments. The present disclosure encompasses embodiments in which parts or elements are omitted. The present disclosure encompasses any replacement or combination of parts or elements between one embodiment and another embodiment. The disclosed technical scope is not limited to the description of the embodiments. The disclosed technical scope is defined by the description of the claims, and should be interpreted as including all modifications within the meaning and scope equivalent to the description of the claims.

The apparatus and techniques described in the present disclosure may be implemented by a special purpose computer comprising a processor programmed to perform one or more functions embodied in a computer program. Alternatively, the apparatus and techniques described in the present disclosure may be implemented using dedicated hardware logic circuitry. Alternatively, the apparatus and techniques described in the present disclosure may be implemented by one or more special purpose computers comprising a combination of a processor executing a computer program and one or more hardware logic circuits. Further, the computer program may also be stored in a computer-readable, non-transitory, tangible recording medium as instructions to be executed by a computer.

### Disclosure of Technical Idea

The description of the present application discloses several technical ideas described in several clauses listed below. Some clauses may be written in a multiple dependent form, with the subsequent clause referring to the preceding clause as an alternative. These clauses written in a multiple dependent form define multiple technical ideas.

### <Technical Idea 1>

A soil sensor includes: a first signal line facing soil to transmit a high-frequency electromagnetic wave; a porous body having a water permeability and a water retention property; a second signal line facing the soil through the porous body to transmit a low-frequency electromagnetic wave having a frequency lower than the high-frequency wave and; and a ground line arranged on an inside of the first signal line. In the soil sensor, the second signal line is arranged on an inside of the first signal line and the ground line.

### <Technical Idea 2>

In the soil sensor according to technical idea 1, the frequency of the electromagnetic waves transmitted through the first signal line is set to a frequency band that is 100 times or more higher than the frequency of the electromagnetic waves transmitted through the second signal line.

### <Technical Idea 3>

The soil sensor according to technical idea 1 or technical idea 2, further includes a processing unit that is configured to measure a volumetric water content of the soil using a transmission signal transmitted through the first signal line as a high-frequency electromagnetic wave, and to measure a water potential of the soil using a low-frequency electromagnetic wave transmitted through the second signal line.

### <Technical Idea 4>

In the soil sensor according to technical idea 3, the processing unit is configured to further measure an electrical conductivity of the soil based on a waveform of the transmission signal transmitted through the first signal line as the high-frequency electromagnetic wave.

### <Technical Idea 5>

In the soil sensor according to any one of technical ideas 1 to 4, a surface shape of the porous body exposed to the soil is a polygon having at least four sides.

### <Technical Idea 6>

The soil sensor according to any one of technical ideas 1 to 5 further includes a base in which the first signal line and the ground line are provided. In the soil sensor, a surface of the base has projections and recesses having a depth of 0.1 to 500 μm.

### <Technical Idea 7>

The soil sensor according to any one of technical ideas 1 to 5 further includes a base in which the first signal line and the ground line are provided. In the soil sensor, a plurality of protrusions are provided on the surface of the base.

### <Technical Idea 8>

The soil sensor according to any one of technical ideas 1 to 5 further includes a base in which the first signal line and the ground line are provided. In the soil sensor, a plurality of the first signal lines are arranged at a plurality of spaced positions in the base.

### <Technical Idea 9>

The soil sensor according to any one of technical ideas 1 to 4 further includes a base in which the first signal line and the ground line are provided. In the soil sensor, the base and the porous body are integrally formed in a regular polyhedron shape.

### <Technical Idea 10>

The soil sensor according to any one of technical ideas 1 to 7 further incudes: a base in which the first signal line and the ground line are provided; a case housing a circuit section electrically connected to the first signal line and the second signal line, and housing a part of the base; and a seal supporter configured to support the base on an outside of the case, and made in a fillet shape to be in close contact with a surface of a portion of the base, protruding outward from the case and the surface of the case.

### <Technical Idea 11>

The soil sensor according to any one of technical ideas 1 to 4 further includes: a base in which the first signal line and the ground line are provided; and a laminated structure in which an outer supporter, the porous body, and the base are laminated in this order from an outside to an inside, and which is restrained by a restraining member. In the soil sensor, a surface of the outer supporter has a flatness with which no gap of more than 2 mm is generated between the outer supporter and the porous body.

### <Technical Idea 12>

The soil sensor according to any one of technical ideas 1 to 5 further includes a base in which the first signal line and the ground line are provided. In the soil sensor, the base includes a surface layer covering the first signal line and the ground line.

### <Technical Idea 13>

In the soil sensor according to technical idea 12, the surface layer includes a plurality of layers laminated from an outside to an inside, and has a glass layer or a ceramic layer on the inside of an outermost resin layer.

### <Technical Idea 14>

The soil sensor according to any one of technical ideas 1 to 13 further includes a processing unit configured to measure a volumetric water content of the soil using a transmission signal transmitted through the first signal line as the high-frequency electromagnetic wave, and to measure a water potential of the soil using the low-frequency electromagnetic wave transmitted through the second signal line. In the soil sensor, the processing unit determines whether an abnormal state exists by using a measured volumetric water content and a measured water potential, and pre-stored two abnormality determination curves that indicate a relationship between the volumetric water content and the water potential and that are stored. Here, the two abnormality determination curves are a first curve indicating a poor adhesion with the soil and a second curve indicating a dryness of soil.

### <Technical Idea 15>

The soil sensor according to any one of technical ideas 1 to 13 further includes a processing unit configured to measure a volumetric water content of the soil using a transmission signal transmitted through the first signal line as the high-frequency electromagnetic wave, and to measure a water potential of the soil using the low-frequency electromagnetic wave transmitted through the second signal line. In the soil sensor, the processing unit determines whether an abnormal state exists based on a result of comparing measurement values at two different positions.

What is claimed is:

1. A soil sensor comprising:
a first signal line facing soil to transmit a high-frequency electromagnetic wave;
a porous body having a water permeability and a water retention property;
a second signal line facing the soil through the porous body to transmit a low-frequency electromagnetic wave having a frequency lower than the high-frequency wave and; and
a ground line arranged on an inside of the first signal line, wherein
the second signal line is arranged on an inside of the first signal line and the ground line.

2. The soil sensor according to claim 1, wherein
the frequency of the electromagnetic wave transmitted through the first signal line is set to a frequency band that is 100 times or more higher than the frequency of the electromagnetic wave transmitted through the second signal line.

3. The soil sensor according to claim 1, further comprising:
a processing unit configured to measure a volumetric water content of the soil using a transmission signal transmitted through the first signal line as a high-frequency electromagnetic wave, and to measure a water potential of the soil using a low-frequency electromagnetic wave transmitted through the second signal line.

4. The soil sensor according to claim 3, wherein
the processing unit is configured to further measure an electrical conductivity of the soil based on a waveform of the transmission signal transmitted through the first signal line as the high-frequency electromagnetic wave.

5. The soil sensor according to claim 1, wherein
a surface shape of the porous body exposed to the soil is a polygon having at least four sides.

6. The soil sensor according to claim 1, further comprising:
a base in which the first signal line and the ground line are provided, wherein
a surface of the base has projections and recesses having a depth of 0.1 to 500 μm.

7. The soil sensor according to claim 1, further comprising:
a base in which the first signal line and the ground line are provided, wherein
a plurality of protrusions are provided on the surface of the base.

8. The soil sensor according to claim 1, further comprising:
a base in which the first signal line and the ground line are provided, wherein
a plurality of the first signal lines are arranged at a plurality of spaced positions in the base.

9. The soil sensor according to claim 1, further comprising:
a base in which the first signal line and the ground line are provided, wherein
the base and the porous body are integrally formed in a regular polyhedron shape.

10. The soil sensor according to claim 1, further comprising:
a base in which the first signal line and the ground line are provided;
a case housing a circuit section electrically connected to the first signal line and the second signal line, and housing a part of the base; and
a seal supporter configured to support the base on an outside of the case, and made in a fillet shape to be in close contact with a surface of a portion of the base, protruding outward from the case and the surface of the case.

11. The soil sensor according to claim 1, further comprising:
a base in which the first signal line and the ground line are provided; and
a laminated structure in which an outer supporter, the porous body, and the base are laminated in this order from an outside to an inside, and which is restrained by a restraining member, wherein
a surface of the outer supporter has a flatness with which no gap of more than 2 mm is generated between the outer supporter and the porous body.

12. The soil sensor according to claim 1, further comprising:
a base in which the first signal line and the ground line are provided, wherein
the base includes a surface layer covering the first signal line and the ground line.

13. The soil sensor according to claim 12, wherein
the surface layer includes a plurality of layers laminated from an outside to an inside, and has a glass layer or a ceramic layer positioned on inside of an outermost resin layer.

14. The soil sensor according to claim 1, further comprising:
a processing unit configured to measure a volumetric water content of the soil using a transmission signal transmitted through the first signal line as the high-frequency electromagnetic wave, and to measure a water potential of the soil using the low-frequency electromagnetic wave transmitted through the second signal line, wherein
the processing unit determines whether an abnormal state exists by using a measured volumetric water content and a measured water potential, and pre-stored two abnormality determination curves that indicate a relationship between the volumetric water content and the water potential and that are stored, and
the two abnormality determination curves are a first curve indicating a poor adhesion with the soil and a second curve indicating a dryness of soil.

15. The soil sensor according to claim 1, further comprising:
a processing unit configured to measure a volumetric water content of the soil using a transmission signal transmitted through the first signal line as the high-frequency electromagnetic wave, and to measure a water potential of the soil using the low-frequency electromagnetic wave transmitted through the second signal line, wherein
the processing unit determines whether an abnormal state exists based on a result of comparing measurement values at two different positions.
